(19) United States
(12) Patent Application Publication
(10) Pub. No.: US 2013/0072978 A1
(43) Pub. Date: Mar. 21, 2013

(54) VERTEBRAL BODY REPLACEMENT AND CERVICAL PLATE CONSTRUCTION

(71) Applicants: Joshua M. AMMERMAN, Bethesda, MD (US); Matthew D. Ammerman, Bethesda, MD (US)

(72) Inventors: Joshua M. AMMERMAN, Bethesda, MD (US); Matthew D. Ammerman, Bethesda, MD (US)

(21) Appl. No.: 13/234,413

(22) Filed: Sep. 16, 2011

(51) Int. Cl. A61B 17/70; A61F 2/44

(52) U.S. Cl. 606/246; 623/17.11

(57) ABSTRACT

A vertebral body replacement (VBR) and cervical plate apparatus for securing the VBR in a desired position in a space between superior and inferior vertebral bodies. The VBR has a predrilled opening in a center portion of its anterior face that is constructed to receive therein a first attachment device. The cervical plate has holes in the upper and lower portions thereof that are constructed to receive second attachment devices for securing the cervical plate to the superior and inferior vertebral bodies in a desired position. The cervical plate has an elongated slot in a central portion thereof that is constructed to receive therethrough in movable relation the first attachment device which is receivable in the predrilled opening in the VBR for securing the cervical plate thereto.

### BACKGROUND OF THE INVENTION

1. Field of the Invention

The present invention relates to a vertebral body replacement and cervical plate apparatus and, more particularly, to such an apparatus for securing a vertebral body replacement in a desired position in a space between superior and inferior vertebral bodies.

2. Detailed Description of the Invention

Resection of either part of or all of a cervical vertebra may be required during the surgical management of a variety of degenerative, neoplastic, congenital and traumatic conditions of the cervical spine. Currently a number of vertebral body replacements (VBR) exist as do a number of cervical plating systems to maintain the position and stability of the spine following vertebral body resection. In addition, hardware has been designed and manufactured that incorporates the vertebral body replacement and plate into a single piece of hardware.

In order for a successful fusion to occur, the VBR, which may be a graft or implant, must be placed into physical contact with and receive pressure from the adjacent vertebrae. Also, the VBR and vertebrae must be stabilized with respect with each other. Previous attempts to stabilize the vertebrae and VBR have had many deficiencies. For example, prior implants have not adequately secured the VBR between the vertebrae or have not properly provided for post-surgery shifting of the space between the vertebrae.

None of the existing technology permits an independent VBR to be fixed to an independent cervical plating system. By rigidly affixing the VBR to the cervical plate, the risk of graft or VBR migration into the spine canal is greatly reduced. Also, such a construction permits easy and reproducible affixation of the cervical plate to the adjacent vertebral bodies exactly over the VBR. Third, if the cervical plate is in a semi-constrained manner, stress shielding is reduced which promotes bone fusion.

### BRIEF SUMMARY OF THE INVENTION

In accordance with the present invention, VBR and cervical plate apparatus is provided for securing the VBR in a desired position in a space between superior and inferior vertebral bodies. The VBR has a predrilled opening in a center portion of its anterior face that is constructed to receive therein a first attachment device. The cervical plate has holes in the upper and lower portions thereof that are constructed to receive second attachment devices for securing the cervical plate to superior and inferior vertebral bodies in a desired position. The cervical plate also has an elongated slot in a central portion thereof that extends between the upper and lower portions thereof. The slot is constructed to receive therethrough in movable relation the first attachment device which is receivable in the predrilled opening in the VBR for securing the cervical plate thereto.

The elongated slot in the cervical plate permits it to be affixed to the superior and inferior vertebral bodies in a desired anatomic position while still permitting the first attachment device to line up with the predrilled opening in the VBR, thereby accommodating each patient's unique vertebral body height/anatomy. The first attachment device and second attachment devices may be of any suitable construction, such as screws, expandable connectors or frictional connectors.

### BRIEF DESCRIPTION OF THE DRAWINGS

FIG. 1 is a front elevational view of a cervical plate constructed in accordance with the present invention;

FIG. 2 is a front elevational view of a VBR constructed in accordance with the present invention; and

FIG. 3 is a front elevational view of the cervical plate of FIG. 1 connected to superior and inferior vertebral bodies and also to the VBR positioned between the vertebral bodies.

### DETAILED DESCRIPTION OF THE INVENTION

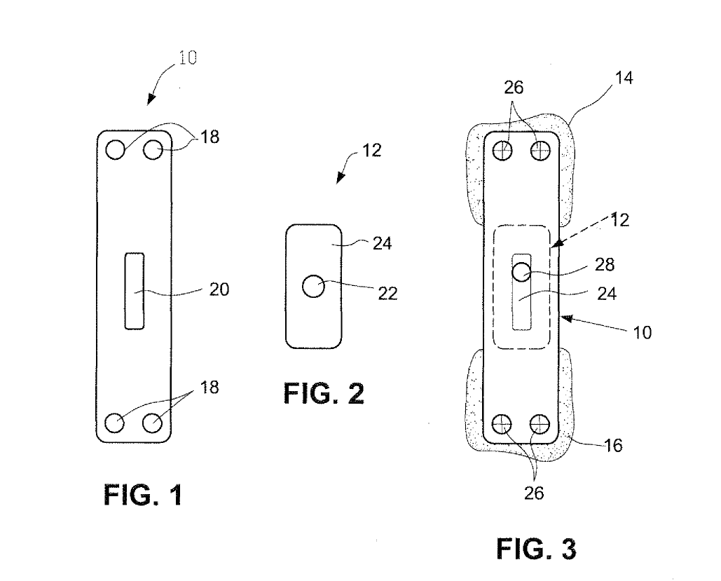

The apparatus of the present invention comprises a combination of the cervical plate 10 shown in FIG. 1 and the VBR shown in FIG. 2 for securing the VBR in a desired position in a space between the superior and inferior vertebral bodies 14 and 16 shown in FIG. 3,

Referring to FIG. 1, the cervical plate 10 may be of any suitable shape such as rectangular and comprises holes 18 near the upper and lower corners thereof for receiving attachment devices therethrough for securing the cervical plate 10 to the superior and inferior vertebral bodies 14 and 16 as shown in FIG. 3. The cervical plate 10 may be formed of any suitable medically acceptable material known to those skilled in the art.

The cervical plate 10 also comprises an elongated slot 20 in a central portion thereof that extends between the upper and lower portions thereof and is of a size to movably receive therein a suitable attachment device for securing the cervical plate to the VBR in the manner shown in FIG. 3. The slot 20 may be of any suitable length such as 1-2 cm.

The VBR 12 shown in FIG. 2 may be of any suitable size and shape for positioning between the superior and inferior vertebral bodies 14 and 16 shown in FIG. 3. Also, the VBR 12 may be formed of any suitable medically acceptable material, e.g., a synthetic material such as polyether ether ketone. The VBR has a predrilled opening 22 in a center portion of its anterior face 24 that is constructed to receive therein the attachment device 28 that is inserted through the elongated slot 20 of the cervical plate 10, as shown in FIG. 3.

The attachment devices 26 that extend through the holes 18 in the cervical plate 10 for securing it to the superior and inferior vertebral bodies 14 and 16 may be of any suitable construction, such as a screw, expandable connector or a frictional connector. Similarly, the attachment device 28 that extends through the elongated slot 20 of the cervical plate 10 and is received in the predrilled opening 22 of the VBR may be of any suitable construction, such as a screw, expandable connector or frictional connector. If a screw is used for the attachment device 28, the interior of the predrilled hole 22 in the VBR 12 would be provided with internal threads that match the threads of the screw.

It will be readily seen that the provision of the elongated slot 20 in the cervical plate 10 and the predrilled hole 22 in the VBR 12 permits the cervical plate 10 to be affixed to the superior and inferior vertebral bodies 14 and 16 in a desired anatomic position while still permitting the attachment device 28 extending through the elongated slot 20 of the cervical plate to be moved to properly line up with the predrilled hole 22 in the VBR to accommodate each patient's unique vertebral body height/anatomy. This construction also prevents stress shielding of the VBR and promotes fusion of the VBR to the adjacent superior and inferior vertebral bodies 14 and 16.

The predrilled opening in the anterior face of the VBR is an important feature for the reason that it would be impossible or impractical to attempt to place an attachment device such as a screw into the VBR without the predrilled opening. The synthetic material of the VBR is too hard to be pierced with a screw or other attachment device or, if bone were used for the VBR, it would likely shatter or split if an attempt were made to pierce it with a screw or other attachment device without a predrilled opening.

The novel construction of the combined VBR and cervical plate apparatus of the present invention has numerous advantages. First, by affixing the cervical plate to the VBR, the risk of migration of the VBR into the spinal canal is greatly reduced. Second, the present construction permits easy and reproducible affixation of the cervical plate to the superior and inferior vertebral bodies exactly over the VBR. Third, by securing the cervical plate to the VBR in a semi-constrained manner, stress shielding is reduced and bone fusion is promoted.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. Vertebral body replacement and cervical plate apparatus for securing a vertebral body replacement in a desired position in a space between superior and inferior vertebral bodies,
the vertebral body replacement having a predrilled opening in a center portion of its anterior face that is constructed to receive therein a first attachment device; and
a cervical plate having holes in upper and lower portions thereof that are constructed to receive second attachment devices for securing the cervical plate to the superior and inferior vertebral bodies in a desired position, the cervical plate having an elongated slot in a central portion thereof that extends between the upper and lower portions thereof, the slot being constructed to receive therethrough in movable relation the first attachment device which is receivable in the predrilled opening in the vertebral body replacement for securing the cervical plate thereto.

2. The apparatus of claim I wherein the predrilled opening in the vertebral body replacement has interior threads, and the first attachment device is a screw having external threads that mate with the interior threads of the vertebral body replacement.

3. The apparatus of claim 2 wherein the second attachment devices are bone screws.

4. The apparatus of claim 1 wherein the cervical plate is rectangular in shape and the holes are located near each corner of the cervical plate.

5. The apparatus of claim I wherein the slot in the cervical plate is approximately 1 cm. to 2 cm. in length.

6. The apparatus of claim 1 wherein the vertebral body replacement has a predetermined shape for fitting in the space between the superior and inferior vertebral bodies.
